(19) United States
(12) Patent Application Publication
(10) Pub. No.: US 2016/0237505 A1
(43) Pub. Date: Aug. 18, 2016

(54) SERUM MIRNAS FOR THE PROGNOSIS OF PROSTATE CANCER

(71) Applicants: THE BOARD OF TRUSTEES OF THE UNIVERSITY OF ILLINOIS, Urbana, IL (US); THE BOARD OF TRUSTEES OF THE LELAND STANFORD JUNIOR UNIVERSITY, Palo Alto, CA (US)

(72) Inventors: Larisa Nonn, Chicago, IL (US); Brittany Mihelich, Chicago, IL (US); Donna Peehl, Stanford, CA (US)

(21) Appl. No.: 15/025,710

(22) PCT Filed: Oct. 15, 2014
(86) PCT No.: PCT/US2014/060642

§ 371 Date: Mar. 30, 2016

Related U.S. Application Data
(60) Provisional application No. 61/891,122, filed on Oct. 15, 2013.
(60) Provisional application No. 61/991,127, filed on May 9, 2014.

(51) Int. Cl. C12Q 1/68

(52) U.S. Cl. CPC C12Q 1/6886; C12Q 2600/158; C12Q 2600/178; C12Q 2600/118; C12Q 2600/112

(57) ABSTRACT

A prostate cancer prognostic serum miRNA panel associated with low-risk prostate cancer is provided that improves prognosis determination and the selection of an appropriate treatment regime. More particularly, the prostate cancer prognostic miRNAs can distinguish between lowgrade and high-grade prostate cancers.

This application claims the benefit of priority of U.S. Provisional Application No. 61/891,122, filed Oct. 15, 2013, and U.S. Provisional Application No. 61/991,127, filed May 9, 2014, the contents of each of which are incorporated herein by reference in their entireties.

This invention was made with government support under grant number RO1 CA121460 and RO1 CA166588 awarded by the National Institutes of Health. The government has certain rights in the invention.

### BACKGROUND

The degree of dedifferentiation of prostate cancer relates to aggressiveness and is reflected by pathologist-determined Gleason grade from 1 to 5, of which 5 is the most dysplastic. Risk of cancer recurrence after radical prostatectomy increases proportionally with the percent of Gleason grade 4+5 in the tumor whereas patients with confirmed 100% Gleason grade 3 cancer rarely recur (Stamey, et al. (1999) JAMA 281:1395-400). Conversely, the multifocal nature of prostate cancer still leads to uncertainty in confident classification of patients with indolent disease as upgrading at the time of prostatectomy still occurs in 30-50% of men with Gleason grade 3 biopsies (Epstein, et al. (2012) European Urology 61:1019-24). There are several pre-surgical risk assessment calculators such as the University of California, San Francisco, Cancer of the Prostate Risk Assessment (UCSF-CAPRA) index (Cooperberg, et al. (2005) J. Urol. 173:1938-42) and the Prostate Cancer Prevention Trial (PCPT) Risk Calculator (Thompson, et al. (2013) New Engl. J. Med. 369:603-10) that incorporate clinical variables to estimate risk of biochemical recurrence (BCR) post-radical prostatectomy. While these calculators are useful for clinical management of prostate cancer patients with the lowest and highest risk, patients with intermediate risk present a clinical challenge.

MicroRNAs (miRNAs) are of use as biomarkers of disease as they can be readily detected and are stable in serum (Moltzahn, et al. (2011) Cancer Res. 71:550-60; Bryant, et al. (2012) Br. J. Cancer 106:768-74). MiRNAs are small, non-coding RNAs that post-transcriptionally regulate gene expression via binding to the 3′ untranslated region of target messenger RNAs (Lau, et al. (2001) Science 294:858-62). In addition to intracellular function, profiling studies have identified cell-free serum miRNAs differentially present in the sera of prostate cancer patients compared to healthy controls (Moltzahn, et al. (2011) Cancer Res. 71:550-60; Brase, et al. (2011) Internat. J. Cancer 128:608-16; Mitchell, et al. (2008) Proc. Natl. Acad. Sci. USA 105:10513-8; U.S. Pat. No. 8,461,315; WO 2010/115833; WO 2011/080315; WO 2014/085906; WO 2010/135692; U.S. Pat. No. 7,642,348; WO 2007/081740; Sita-Lumsden, et al. (2013) Br. J. Cancer 108:1925-30) and miRNAs that differ in prostate cancer patients with metastases (Bryant, et al. (2012) Br. J. Cancer 106:768-74). However, there is little overlap between the miRNAs identified in these studies, which may be a result of differences in study design, methodologies, and small numbers of subjects (Sapre & Selth (2013) Prostate Cancer 2013:539680).

### SUMMARY OF THE INVENTION

The present invention provides kits and methods for use in the prognosis of prostate cancer. In particular, the invention provides a method for determining the aggressiveness of prostate cancer in a patient by (a) measuring, in a serum sample from a patient who is suspected of having or has been diagnosed with prostate cancer, the expression level of miRNAs consisting of one or more of miR-24, miR26b, miR-30c, miR-93, miR-106a, miR-223, miR-451, miR-1207-5p, miR-874, miR-1274a, miR-141, miR-107, miR-130b, miR-183, miR-96, miR-182, miR-146a, miR-125b and miR-100; and (b) comparing the measured level of expression of the miRNAs from the patient with a pre-determined threshold or standard curve, wherein the level of expression of the one or more miRNAs as compared to the pre-determined threshold or standard curve is indicative of the patient having a low-risk of aggressive disease. In some embodiments, the measured level of expression of the miRNAs is normalized to one or more control RNAs, e.g., Sp3, Sp6 or cel-miR-39. In further embodiments, the level of expression of the miRNAs is measured via reverse transcription-quantitative PCR with primers having sequences specific to the miRNAs. In yet other embodiments, the patient having a low-risk of aggressive disease is treated with active surveillance.

The kit of the present invention includes a pre-determined threshold or standard curve of miRNA expression levels, which are indicative of benign prostatic hyperplasia or low-risk prostatic cancer, wherein the miRNAs consist of one or more of miR-24, miR26b, miR-30c, miR-93, miR-106a, miR-223, miR-451, miR-1207-5p, miR-874, miR-1274a, miR-141, miR-107, miR-130b, miR-183, miR-96, miR-182, miR-146a, miR-125b and miR-100; and one or more control RNAs, e.g., Sp3, Sp6 or cel-miR-39.

### BRIEF DESCRIPTION OF THE DRAWINGS

FIG. 1 shows circulating miRNAs levels in patients with benign prostatic hyperplasia (BPH), low-grade prostate cancer and high-grade prostate cancer. Waterfall plots of RQ values (y-axis) are shown for let-7a, miR-24, 26a, 30c, 93, 100, 103, 106a, 107, 130b, 146a, 223, 451 and 874, wherein each patient is on the x-axis sorted by RQ within each group and by disease. BPH=benign prostate hyperplasia (N=50). Gleason 3=100% of the tumor was Gleason grade 3 (N=50). Gleason 4+5=30-90% of the tumor was Gleason 4+5 (N=50). *p<0.05 compared to Gleason 3 and ** p<0.05 compared to BPH by Wilcoxon rank sum test. A waterfall plot ranks all the RQs within each group to visualize differences. RQ is the relative quantification of the miRNA levels after normalization to spike-in quality controls (Sp3, Sp6, cel-miR-39) and to endogenous control of RNA input.

FIG. 2 shows pretreatment circulating miRNAs levels in prostate cancer patients according to future BCR (biochemical/PSA recurrence). Waterfall plots of let-7a, miR-26b, 103, 106a, 107, 130b, 223 and 451 levels measured by RT-qPCR in serum from patients (N=100) with and without BCR. *p<0.05 by Wilcoxon rank sum test.

FIG. 3 shows that circulating miRNAs predict disease-free survival. Kaplan-Meier survival curve for all prostate cancer patients (N=100) above (high score) and below (low score) the miR Risk Score threshold.

### DETAILED DESCRIPTION OF THE INVENTION

Approximately 50% of prostate cancers diagnosed in population-based studies show the pathological features of the incidental cancers found at autopsy (Gosselaar, et al. (2005) BJU Int. 95:231-7). This indicates that a subset of men diagnosed with prostate cancer do not require any active, invasive treatment. Indeed, in the population-based European Randomized study of Screening for Prostate Cancer (ERSPC; Schroder, et al. (2009) N. Engl. J. Med. 360:1320-8), over 600 men with clinically and pathologically defined low risk prostate cancer were observed without primary treatment for over a period of ten years (Roemeling, et al. (2007) Eur. Urol. 51:1244-50), wherein overall survival was documented at 70%, and wherein none died of prostate cancer. According, the diagnosis of such low-risk or indolent cancers, without the need for a prostate biopsy would reduce the morbidity associated with prostate cancer overtreatment.

It has now been found that circulating miRNAs are stable serum markers that can distinguish prostate cancer patients with and without aggressive disease. The serum levels of 21 miRNAs were measured in men with benign prostatic hyperplasia (BPH), low-grade prostate cancer (100% Gleason grade 3), and high-grade prostate cancer (30-90% Gleason grade 4+5) (N=50 each). Fourteen miRNAs including Let-7a, miR-26b, miR-93, miR-103, miR-107, miR-146a, miR451, miR-24, miR-30c, miR-100, miR-106a, miR-130b, miR-223, and miR-875, were consistently lower in the sera from men with high-grade prostate cancer compared to patients with low-grade prostate cancer or BPH, who had heterogeneous and higher levels of the miRNAs. A combined miR Score had a positive predictive value (PPV) of 0.939 to predict absence of high-grade prostate cancer among prostate cancer and BPH patients. Biochemical recurrence (BCR) was known for the prostate cancer patients and a combined miR Risk Score accurately classified a subset of patients with low risk of BCR (PPV 0.941). Accordingly, the measurement of the serum miRNAs described herein has pre-surgical and/or pre-biopsy utility in combination with clinical risk calculators to distinguish low-risk from high-risk prostate cancer.

Therefore, the present invention provides kits and methods for determining the aggressiveness of prostate cancer in a patient suspected of having prostate cancer or positively diagnosed as having prostate cancer (e.g., by positive biopsy) by measuring the level of expression of prostate cancer prognostic serum miRNAs. “Prostate cancer” refers to cancer arising from the tissue of the prostate gland. Classification systems such as the Gleason score are typically used to classify primary prostate tumors (see, AJCC Cancer Staging Manual. (2010) 7th ed. New York, N.Y., Springer). A tumor with a low Gleason score typically grows slowly and may not pose a significant immediate threat to a patient such that the patient may be monitored by active surveillance. “High-risk” or “high-grade” prostate cancer includes aggressive disease (e.g., Gleason score greater than 4 or 5). High-risk patients are more likely to have recurrent prostate cancer, which refers to local or distant recurrence of prostate cancer. A prostate cancer can recur locally in the tissue next to the prostate or in the seminal vesicles, affect the surrounding lymph nodes in the pelvis or lymph nodes, or spread to tissues or organs next to the prostate. “Low-risk” or “low-grade” prostate cancer includes cancer with a Gleason score of 3 for which monitoring is appropriate.

A prostate cancer prognostic serum miRNA panel, as disclosed herein, is a panel of miRNAs that differentiates low-risk prostate cancer or BPH from high-risk prostate cancer. The miRNAs of the panel are therefore of use in identifying patients for whom aggressive treatment is not needed. The prostate cancer prognostic serum miRNA panel of this invention includes miR-93, miR-451, miR-24, miR-106a, miR-223, Let7a, miR-26b, miR-30c, miR-100, miR-103, miR-107, miR-130b, miR-146a and miR-874. The sequences of these miRNAs are publically available, for example, in The miRNA Registry (Griffiths-Jones (2004) Nucleic Acids Res. 32:D109-D111) and GENBANK.

The step of measuring miRNA levels in a serum sample can be carried out using any suitable method for detecting, measuring, assaying, screening or quantifying miRNAs. Examples of such methods include, without limitation, methods based on hybridization analysis of polynucleotides, methods based on sequencing of polynucleotides, and methods based on cloning or molecular barcoding (e.g., NANOSTRING as disclosed in U.S. Pat. No. 7,473,767). Particular examples of methods based on hybridization analysis include, without limitation, northern blotting, RNA expression assays such as microarray analysis and in situ hybridization (Parker & Barnes (1999) Meth. Mol. Biol. 106:247-283); RNAse protection assays (Hod (1992) BioTechniques 13:852-854); PCR-based methods such as reverse transcription PCR (RT-PCR) (Weis, et al. (1992) Trends Genet. 8:263-264), RT-qPCR, and in situ PCR. Methods to profile gene expression may also employ antibodies that can recognize sequence-specific duplexes such as RNA duplexes and DNA-RNA hybrid duplexes. Examples of methods based on sequencing include without limitation, serial analysis of gene expression (SAGE), deep sequencing (Creighton, et al. (2009) Brief Bioinform. 10(5):490), RNA sequencing and gene expression analysis by massively parallel signature sequencing (MPSS).

When using PCR-based methods and techniques to detect, measure, assay, screen or quantify the level of a miRNA, the miRNA of the prostate cancer prognostic serum miRNA panel are amplified. In particular, RT-PCR methods are known in the art and for example are described in US 2012/0028264, US 2012/0142753, US 2012/0157344 and US 2012/0088687. RT-PCR can be performed using commercially available kits and equipment in accordance with the manufacturer's instructions (e.g., the GeneAmp RNA PCR kit; Perkin Elmer, Calif.). Similarly, primers or probes, as well as arrays of primers and probes, can be generated based upon the sequence of the miRNA or obtained from commercial sources, e.g., EXIQON.

A method of the invention can also be carried out by combining isolated miRNA with reagents used in the conversion of miRNA to cDNA; treating the converted cDNA with amplification reaction reagents (such as cDNA PCR reaction reagents) in a container along with an appropriate mixture of nucleic acid primers; reacting the contents of the container to produce amplification products; and analyzing the amplification products to detect the presence of miRNA in the sample. The analyzing step may be accomplished using northern blot analysis to detect the presence of miRNA using, e.g., the probes. The analysis step may be further accomplished by quantitatively detecting the presence of miRNAs in the amplification product, and comparing the quantity of miRNAs detected against a panel of expected values for the known presence or absence of the miRNAs in control samples derived using similar primers.

RT-PCR may be performed using an internal standard or reference. In some methods the reference is a miRNA that is expressed at quite constant levels among samples from low- or high-risk prostate cancer patients, and is expressed at a quite constant level among the same sample taken from different patients. miRNA expression levels may be normalized relative to the mean of one, two, three, four, five, or six or more reference miRNAs. A normalized reference expression measurement may be from 2 to 15 where each unit increase reflects a 2-fold increase in miRNA level or quantity. In some embodiments, RT-qPCR is used to measure the expression level of the miRNAs disclosed herein.

High throughput methods can also be used to measure the expression of multiple miRNAs. Microarrays are prepared by selecting probes and immobilizing them to a solid support or surface which may be either porous or non-porous. For example, the probes can be attached to a nitrocellulose or nylon membrane or filter covalently at either the 3′ or the 5′ end of the polynucleotide probe. The solid support may be a glass or plastic surface. In this respect, a microarray of this invention can include a support or surface with an ordered array of hybridization sites or probes, each capable of hybridizing to one of the miRNAs disclosed herein. The microarrays can be addressable arrays, and in particular positionaly addressable arrays. Each probe of the array is typically located at a known, predetermined position on the solid support such that the identity of each probe can be determined from its position in the array. Preferably, each probe is covalently attached to the solid support at a single site.

Described herein are miRNA molecules that are differentially expressed in patients having prostate cancer with a Gleason score of 4+5 compared with patients having prostate cancer with a Gleason score of 3 or patients with benign prostatic hyperplasia. Therefore, measuring the expression of one or more of the differentially expressed miRNAs in a patient can distinguish low-risk versus high-risk disease. High levels of miR-93, miR-451, miR-24, miR-106a, miR-223, Let7a, miR-26b, miR-30c, miR-100, miR-103, miR-107, miR-130b, miR-146a and miR-874 expression are only observed in patients with BPH or low-grade PCA and never in high-grade PCa. Therefore, patients expressing high levels of these miRNA as compared to a threshold level or standard curve would benefit from treatment with active surveillance.

Once the levels of expression of the miRNAs of the prostate cancer prognostic serum miRNA panel are measured, the measured level is compared with a threshold level or standard curve, e.g., from one or more control patients. Control or standard levels of expression of the prostate cancer prognostic serum miRNA panel can be any reference or predetermined threshold value. In some aspects, the control is a value from a sample obtained from a plurality of patients with high-risk prostate cancer. In other embodiments, the threshold level is a historical value (i.e., a previously tested control sample or group of samples representing baseline). In further embodiments, the control is a standard curve representing the average value or average range of values obtained from a plurality of patient samples (such as an average value or range of values of miRNAs of the panel). A threshold or standard may be in the form of a graph or table that permits comparison of measured experimentally determined values.

The results of a patient's prognostic screen is typically displayed or provided to a user such as a clinician, health care worker or other caregiver, laboratory personnel or the patient. The results may be quantitative information (e.g. the level or amount of a miRNA) or qualitative information (e.g., diagnosis of low-risk disease). The output can include guidelines or instructions for interpreting the results, for example, numerical or other limits that indicate the presence or absence of low-risk disease. The guidelines may also specify the diagnosis, for example whether low-risk disease is present or absent. The output can include tools for interpreting the results to arrive at a diagnosis prognosis or treatment plan, for example, an output may include ranges or cutoffs for abnormal or normal status to arrive at a prognosis or treatment plan. The output can also provide a recommended therapeutic plan, and it may include other clinical information and guidelines and instructions for interpreting the information.

Devices known in the art can be used to transmit the results of a method of the invention. Examples of output devices include without limitation, a visual output device (e.g., a computer screen or a printed paper), an auditory output device (e.g., a speaker), a printer or a patient's electronic medical record. The format of the output providing the results and related information may be a visual output (e.g., paper or a display on a screen), a diagram such as a graph, chart or voltametric trace, an audible output (e.g., a speaker) or a numerical value. In one embodiment, the output is a numerical value, in particular the amount or relative amount of at least one miRNA in a patient's sample. In another embodiment, the output is a graph that indicates a value, such as an amount or relative amount, of the at least one miRNA in the sample from the patient on a standard curve. In a further embodiment, the output (such as a graphical output) shows or provides a cut-off or threshold value or level that indicates the presence of low-risk disease. An output may be communicated to a user by physical, audible or electronic means, including mail, telephone, facsimile transmission, email or an electronic medical record.

The predictive ability of the miRNAs of the invention is exceedingly informative for patients with high levels of the miRNAs, which are present only in the men with BPH, low-grade prostate cancer and no BCR. Accordingly, in lieu of prescribing aggressive treatment options such as surgery, radiation therapy, hormone therapy, chemotherapy, cryotherapy, and/or high-intensity focused ultrasound, a subject a low-risk would be treated with active surveillance thereby reducing the cost and potential morbidity due to overtreatment of prostate cancer.

A kit for carrying out the method of this invention typically includes information concerning a predetermined threshold level or standard curve showing the expression of the miRNAs of the prostate cancer prognostic serum miRNA panel. In addition, the kit includes quality controls for RNA quality, RNA extraction, cDNA synthesis and PCR efficiency. Such quality controls typically include control RNAs, which are exogenous molecules that are spiked into test samples prior to each step to control for variation in RNA extraction (e.g., cel-miR-39), cDNA synthesis (e.g., Sp6) and inter-plate calibration (e.g., Sp3). Accordingly, in certain embodiments, the kit includes cel-miR-39, Sp6 and Sp3 as control RNA.

It is contemplated that the method and kit described herein can be used in combination with a variety of other diagnostic or prognostic factors associated with prostate cancer, in particular associated with biochemical failure, aggressive disease and/or recurrence of prostate cancer. For example, PSA, stage, prostate capsule invasion, surgical margin status, seminal vesicle involvement, lymph node involvement, IL-1a, IP-10 level and/or Gleason grade can be used in combination with the instant kit and method.

The method and kit of the invention are particularly useful following a positive biopsy to identify those patients with high levels of the miRNAs who have a very low risk of harboring high-grade prostate cancer. This risk information is highly valuable because active surveillance or delayed treatment may well be appropriate and confidently recommended for these patients with low-grade disease and a low risk of BCR.

The following non-limiting examples are provided to further illustrate the present invention.

### EXAMPLE 1

### Materials and Methods

Patients and Specimens. Sera were obtained with informed consent under an IRB-approved protocol at Stanford University 1998-2004 and stored at −80° C. Sera from 100 men with prostate cancer were obtained during the pre-operative consultation several days prior to surgery. Patient characteristics and clinicopathologic variables were obtained from an existing database (Table 1). Patient race was not recorded, but the patient population at Stanford during that time period was ˜97% Caucasian. For BPH patients, sera from 50 men were obtained during office visits for evaluation of BPH during this same time period. Absence of prostate cancer in BPH patients was confirmed by at least two sets of ultrasound-guided needle biopsies.

TABLE 1

| | | Prostate Carcinoma Patients | | |
|---|---|---|---|---|
| | | 100% | 30-90% | |
| | | Gleason | Gleason | |
| | BPH | Grade 3 | Grade 4 + 5 | P Value |
| Total | 50 | 50 | 50 | |
| Age | | | | |
| Range | 56-80 | 46-75 | 46-78 | |
| Mean | 66^{a} | 62^{a} | 64 | 0.002 |
| PSA | | | | |
| Range | 1.33-23.31 | 1.2-35.8 | 2.62-490 | |
| Mean | 9.9 | 7.9 | 24.7 | |
| Stage, | | | | |
| Clinical^{b} | | | | |
| T1c | N/A | 33 | 25 | |
| T2a | N/A | 11 | 9 | |
| T2b | N/A | 5 | 12 | |
| T2c | N/A | 1 | 4 | 0.0127 |
| Gleason Sum | | | | |
| Biopsy^{c} | | | | |
| Range | N/A | 6-7 (n = 30) | 6-9 (n = 39) | |
| Mean | N/A | 6.04 | 7.1 | |
| Gleason 7 | | (3 + 4 n = 1) | (3 + 4 n = 10) | |
| | | | (4 + 3 n = 9) | 8.66 × 10⁻¹³ |
| Biochemical | | | | |
| Recurrence^{d} | | | | |
| No | N/A | 50 | 23 | |
| Yes | N/A | 0 | 27 | 1.13 × 10⁻¹⁰ |

^{a}p = 0.002, two-tailed t-test.
^{b}Gleason 3 and Gleason 4 + 5 groups, p = 0.0127 by Fisher Exact Test.
^{c}Gleason 3 and Gleason 4 + 5 groups, p = 8.7 × 10⁻¹³, two-tailed t-test.
^{d}Gleason 3 and Gleason 4 + 5 groups, p = 1.1 × 10⁻¹⁰, Fisher Exact Test.
BPH, Benign Prostatic Hyperplasia.

RNA Extraction, Reverse Transcription and qPCR. Serum samples were coded, blinded and the RNA was extracted from 250 μl of serum using a modified miRNeasy (Qiagen, Valencia, Calif.) protocol. The RNA (4 μl) was reverse-transcribed using a Universal cDNA Synthesis Kit (Exiqon Inc., Denmark). An exogenous oligonucleotide was spiked into the samples prior to each step to control for variation in RNA extraction (cel-miR-39), cDNA synthesis (Sp6) and inter-plate calibration (Sp3). qPCR was run on the VIIA 7 Real-Time PCR System (Applied Biosystems, Foster City, Calif.) using SYBR green and custom Pick and Mix microRNA PCR plates containing primers for 21 miRNAs of interest (Table 2) and the controls Sp3, Sp6, and cel-miR-39 (Exiqon, Inc.). Wells with Ct>37 or poor melting curves were excluded from analysis.

TABLE 2

Results of This Study

| miRNA | Gleason 3 vs Gleason 4 + 5 | BCR |
|---|---|---|
| miR-1274a¹ | ND | ND |
| miR-141² | | |
| miR-182³ | | |
| miR-183³ | | |
| miR-96³ | | |
| Let7a⁴ | p < 0.05 | p < 0.05 |
| miR-103⁵ | | |
| miR-107² | | |
| miR-130b² | | |
| miR-106a¹ | | |
| miR-26b¹ | | |
| miR-451¹ | | |
| miR-223¹ | | |
| miR-93¹ | | p > 0.05 |
| miR-24¹ | | |
| miR-30c¹ | | |
| miR-874¹ | | |
| miR-100⁶ | | |
| miR-146a⁶ | | |
| miR-125b⁶ | p > 0.05 | |
| miR-1207-5p¹ | | |

¹Moltzahn, et al. (2011) Cancer Res. 71: 550-60.
²Bryant, et al. (2012) Br. J. Cancer 106: 768-747.
³Mihelich, et al. (2011) J. Biol. Chem. 286: 44503-11.
⁴Henegan, et al. (2010) Annals Surg. 251: 499-505.
⁵Kan, et al. (2012) BMC Cancer 12: 627.
⁶Giangreco, et al. (2013) Cancer Prevent. Res. 6: 483-94

Normalization of PCR Data. Ct values were first normalized to the Sp3 spike-in for plate-to-plate PCR variability. To control for cDNA syntheses and RNA extraction efficiencies, Ct values were further normalized to Sp6 and cel-miR-39, respectively. Finally, samples were normalized to the total RNA input. Between the analysis groups, there were no statistical differences in the exogenous controls or RNA inputs. EXIQON-recommended endogenous normalizers were significantly different between groups and not utilized. Relative quantities (RQ) were calculated by the ΔΔCt method (Schmittgen & Livak (2008) Nat. Protoc. 3:1101-8).

Differences Between Groups. Samples were un-blinded and RQs were log2-transformed to approximate a normal distribution across individuals. All analyses were performed in R statistical package. ANOVA and Wilcoxon rank sum tests were used to assess differences between groups.

miR Scores for Gleason Grade. All miRNAs that were found to differ between BPH, Gleason grade 3 and Gleason grade 4+5 groups were incorporated into miR Scores. Log odds ratios representing the change in natural log odds of Gleason grade 3, grade 4+5 or BPH status per 2-fold increase in miRNA expression level were calculated by logistic regression. These estimated log odds ratios were then multiplied by the log2 RQ levels for each miRNA in each patient. These analyses resulted in the two miR Scores; miR Scorel, which was optimized to separate low-grade from high-grade prostate cancer patients, and miR Score2, which was optimized to separate BPH and low-grade prostate cancer from high-grade prostate cancer. The equations for the miR Scores, in which the miRNA name represents the log2 RQ value: miR Scorel=(0.269×let-7a)+(0.257×miR-103)+(0.254×miR-451)+(0.255×miR-24)+(0.252×miR-26b)+(0.255×miR-30c)+(0.221×miR-93)+(0.253×miR-106a)+(0.274×miR-223)+(0.188×miR-874)+(0.200×miR-146a)+(0.118×miR-100)+(0.276×miR-107)+(0.204×miR-130b). miR Score2=(0.289×level Let7a)+(0.286×level miR-103)+(0.308×level miR-451)+(0.267×level miR-24)+(0.256×level miR-26b)+(0.282×level miR-30c)+(0.231×level miR-93)+(0.263×level miR-106a)+(0.293×level miR-223)+(0.161×level miR-874)+(0.227×level miR-146a)+(0.165×level miR-125b)+(0.116×level miR-100)+(0.295×level miR-107) (0.183×level miR-130b).

The positive predictive value (PPV) and negative predictive value (NPV) across all possible thresholds were estimated (using each ranked miR Score as a threshold). The miR Score thresholds were cross-validated by two methods.

First, leave-one-outcross-validation (LOOCV) was run. Briefly, one patient at a time was removed from the data set, and then log odds ratios were re-estimated for each miRNA in the remaining patients. The ability of the new miR Score to properly classify the removed patient was then assessed.

Two-fold cross-validation (2-fold CV) approach was also used in which each patient was randomly assigned to a training or validation set of equal size (n=50). Log odds ratios were estimated in the training set and the PPV and NPV of miR Scores were assessed in the validation set based on these estimates. This random sampling was repeated and PPV and NPV were estimated in 10,000 independent iterations.

To assess the ability of the miR Score to distinguish Gleason grade 3 or BPH patients from those with grade 4+5 cancer across the range of scores, a Receiver-Operator Characteristic curve was generated using the software ‘pROC’ (Robin, et al. (2011) BMC Bioinformatics 12:77).

miR Risk Score for BCR. A similar approach as above was used to identify and test the performance of a miR Risk Score for BCR. Briefly, the log odds ratio of risk of BCR was estimated for each significantly associated miRNA and these values were used to calculate a miR Risk Score using the equation: miR Risk Score=(0.223×miR-451)+(0.225×miR-106a)+(0.217×miR-223)+(0.215×miR-107)+(0.147×miR-130b)+(0.217×of let-7a)+(0.196×of miR-26b). A threshold of the PPV and NPV for BCR-free survival was determined and cross-validated as described above.

Kaplan-Meier Analysis. BCR-free survival times were examined in the 100 prostate cancer patients above and below the threshold miR Risk Score of 3.26. Kaplan-Meier curves were plotted using the packages ‘KMsurv’ and ‘survival’ for all patients and those with CAPRA-intermediate status. Patients above and below the mean were compared using log rank tests.

Association of miRNA with CAPRA. Differences between plates were monitored by the expression of Cel-miR-39. Plates were first normalized to each other based on the expression of Sp3 for every sample. Samples were then normalized for extraction efficiency based on Cel-miR-39. Finally, samples were normalized to the expression of let-7a. Relative quantities for each miRNA were estimated for each individual, and samples with expression below 0.01 were excluded. Expression level estimates were log2-transformed to approximate a normal distribution across individuals. All analyses were performed in R statistical package (R-Development-Core-Team. (2008) Vienna, Austria: R Foundation for Statisical Computing). ANOVA and pair-wise t-tests were used to assess differences in expression levels between groups (BPH, 100% Gleason 3 and 30-90% Gleason 4+5). False discovery rates were estimated using the Benjamini-Hochberg method (Hochberg & Benjamini (1990) Stat. Med. 9:811-8). To identify miRNAs that could improve upon the predictive ability of CAPRA, an Akaike Information Criteria (AIC)-based model selection algorithm implemented in the software ‘bestglm’ (McLeod & Changjiang (2011) Bestglm: Best Subset GLM, vol. R package version 0.33) was used. Briefly, this involved comparing the explanatory value and complexity (summarized by AIC) of logistic regression models with all possible combinations of CAPRA and miRNAs as predictors of recurrence. To assess the performance of logistic regression models, ‘epicalc’ (Chongsuvivatwong (2012) Epicalc: Epidemiological Calculator, vol. R package version 2.15.1.0) software was used to generate receiver-operator characteristic (ROC) curves, which were summarized by calculating the area under the curve (AUC). To perform a statistical test of the hypothesis that adding miRNAs to CAPRA increases the AUC, confidence intervals were estimated for AUCs using bootstrap analyses (10⁵ bootstrap replicates) as implemented in the software ‘pROC’(Robin, et al. (2011) BMC Bioinform. 12:77).

### EXAMPLE 2

### Circulating miRNA Levels Differentiate Prostate Cancer Patients

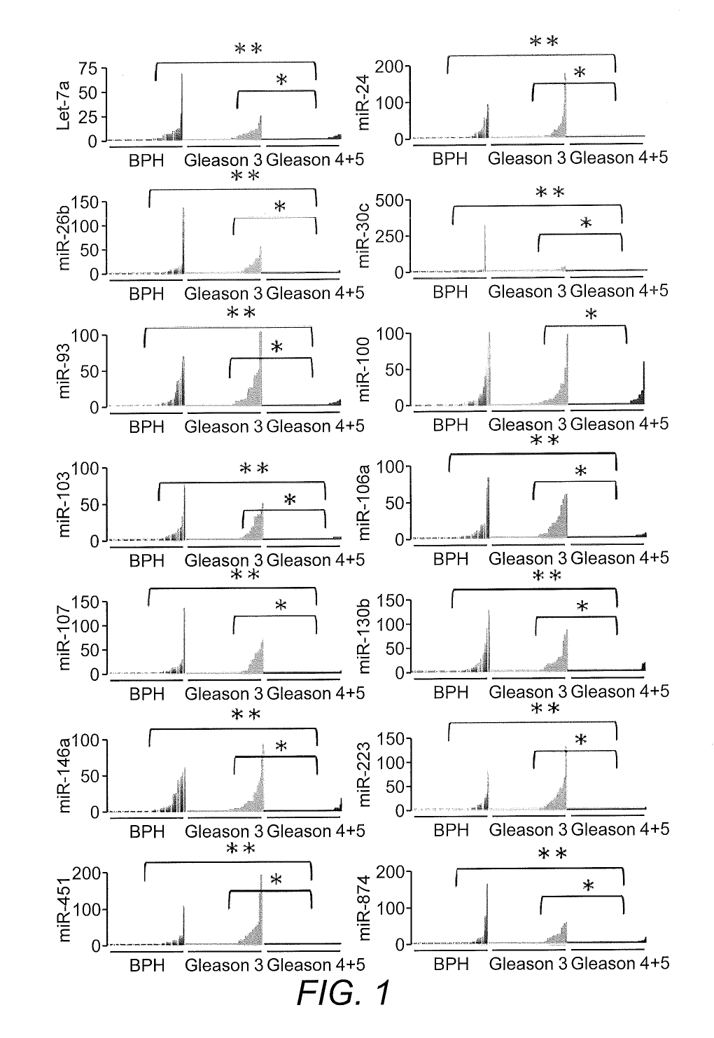

RNA was isolated from sera from 50 men with 100% Gleason grade 3 prostate cancer (low-grade), 50 with 30-90% Gleason grade 4+5 prostate cancer (high-grade), and 50 with BPH (Table 1). The levels of 21 miRNAs, which have been suggested to exhibit differential levels in the sera or cancer tissues of prostate cancer patients compared to non-cancer controls (Table 2)(Moltzahn, et al. (2011) Cancer Res. 71:550-60; Bryant, et al. (2012) Br. J. Cancer 106:768-747; Kan, et al. (2012) BMC Cancer 12:627; Giangreco, et al. (2013) Cancer Prevent. Res. 6:483-94; Mihelich, et al. (2011) J. Biol. Chem. 286:44503-11; Heneghan, et al. (2010) Annals Surg. 251:499-505), were measured using custom PCR EXIQON arrays. Sixteen miRNAs were detectable in the sera and 14 of the miRNAs were detected at uniformly low levels in all of the patients with high-grade prostate cancer, whereas these miRNAs were present at significantly higher and more heterogeneous levels in patients with low-grade Prostate cancer or BPH (FIG. 1). None of the miRNAs were associated with gross hemolysis of the sera.

Both the prognostic and diagnostic values of the miRNAs were examined by calculating a “miR Score” that included the RQ and odds-ratio for each of the miRNAs present at different levels between the groups. For prognosis, miR Scorel distinguished low-grade from the high-grade prostate cancer patients. The threshold of 7.19 had a PPV of 1 and NPV of 0.588, thus highly predictive of low-grade prostate cancer. LOOCV and 2-fold CV showed similar results with PPVs of 0.938 and 1 (SE=0.15), respectively, and NPVs of 0.583 and 0.564 (SE=0.06), respectively, for low-grade prostate cancer. The AUC for low-grade prostate cancer was 0.69 across the range of miR risk scores. For diagnosis, BPH and low-grade prostate cancer patients were examined as one group and compared to high-grade prostate cancer patients using a “miR Score2” that included the same miRNAs as miR Score1 plus miR-125b. A threshold of 7.85 for miR Score2 was highly predictive of BPH or low-grade prostate cancer, with a PPV of 1 and NPV of 0.413. Cross validation of the miR Score by LOOCV showed a PPV=0.938 and NPV=0.366 for absence of high-grade prostate cancer. Similar results were observed with 2-fold CV with a PPV=1 (SE=0.14) and NPV=0.564 (SE=0.04). The AUC for absence of high-grade disease was 0.69 across the range of miR risk scores.

These results indicate that high serum levels of these miRNAs have clinical utility in a risk calculator to identify patients with low risk of harboring high-grade cancer at diagnosis.

### EXAMPLE 3

### miRNAs Levels Predict Disease-Free Survival

miRNAs levels in prostate cancer patients with and without BCR were also examined. Eight of the miRNAs were significantly lower in the prostate cancer patients who had BCR compared to those who did not (FIG. 2). To test these miRNAs as a pre-surgical predictor of BCR, a miR Risk Score was calculated. PPVs were calculated across the range of risk scores to identify an optimal threshold of 3.26 that was highly predictive of disease-free survival (no BCR). LOOCV (PPV=0.941, NPV=0.313) and 2-fold CV (PPV=1.0±0.088, NPV=0.321±0.05) showed similar results. Across all values for the miR Risk Score, the AUC for BCR-free survival was 0.668.

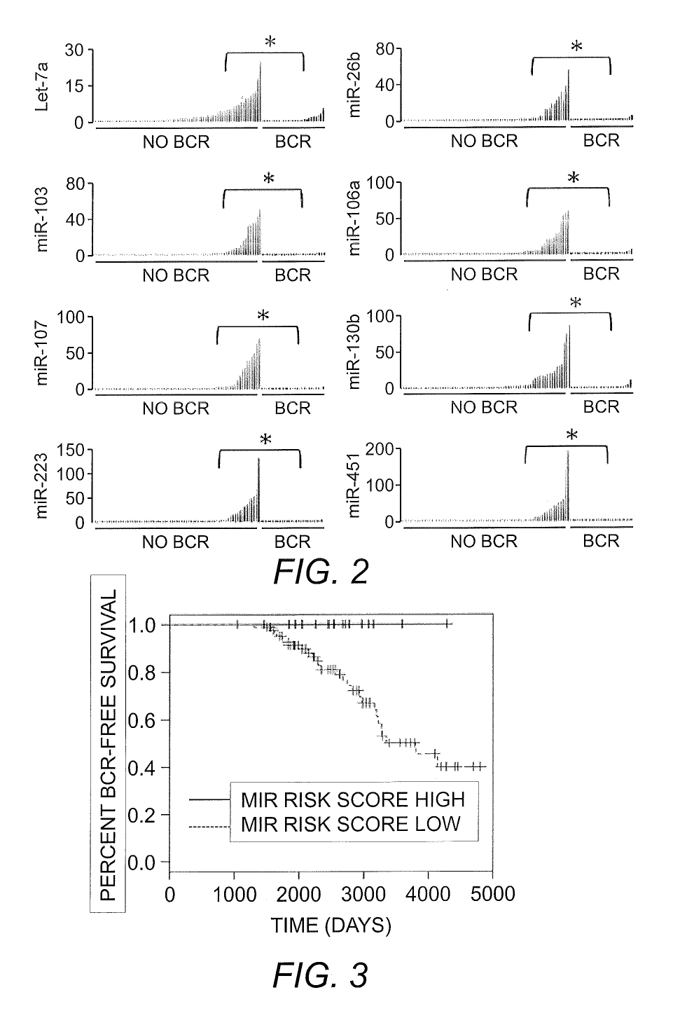

The ability of the miR Risk Score to differentiate time to BCR was examined by Kaplan-Meier curves. A “high score” or “low score” above or below the threshold predicted disease-free survival when examined in all 100 prostate cancer patients (p=0.031, FIG. 3).

In summary, the miR Risk Score was able to identify a subset of prostate cancer patients with a very low risk of BCR, similar to the ability of the miR Score to distinguish men with BPH or low-grade prostate cancer from those with high-grade Prostate cancer.

What is claimed is:

1. A method for determining the aggressiveness of prostate cancer in a patient comprising
(a) measuring, in a serum sample from a patient who is suspected of having or has been diagnosed with prostate cancer, the expression level of miRNAs consisting of one or more of miR-24, miR26b, miR-30c, miR-93, miR-106a, miR-223, miR-451, miR-1207-5p, miR-874, miR-1274a, miR-14l, miR-107, miR-130b, miR-183, miR-96, miR-182, miR-146a, miR-125b and miR-100; and
(b) comparing the measured level of expression of the miRNAs from the patient with a pre-determined threshold or standard curve, wherein the level of expression of the one or more miRNAs as compared to the pre-determined threshold or standard curve is indicative of the patient having a low-risk of aggressive disease.

2. The method of claim 1, wherein the measured level of expression of the miRNAs is normalized to one or more control RNAs.

3. The method of claim 2, wherein the control RNAs comprise Sp3, Sp6 or cel-miR-39.

4. The method of claim 1, wherein the level of expression of the miRNAs is measured via reverse transcription-quantitative PCR with primers having sequences specific to the miRNAs.

5. The method of claim 1, further comprising treating the patient having a low-risk of aggressive disease with active surveillance.

6. A kit for prostate cancer prognosis comprising
(a) a pre-determined threshold or standard curve comprising miRNA expression levels, which are indicative of benign prostatic hyperplasia or low-risk prostatic cancer, wherein the miRNAs consist of one or more of miR-24, miR26b, miR-30c, miR-93, miR-106a, miR-223, miR-451, miR-1207-5p, miR-874, miR-1274a, miR-141, miR-107, miR-130b, miR-183, miR-96, miR-182, miR-146a, miR-125b and miR-100; and
(b) one or more control RNAs.

7. The kit of claim 6, wherein the one or more control RNAs comprise Sp3, Sp6 or cel-miR-39.
